(19) United States
(12) Patent Application Publication
(10) Pub. No.: US 2025/0352553 A1
(43) Pub. Date: Nov. 20, 2025

(54) TREATMENT OF HOSPITAL-ACQUIRED BACTERIAL PNEUMONIA AND VENTILATOR-ASSOCIATED BACTERIAL PNEUMONIA CAUSED BY SUSCEPTIBLE ISOLATES OF ACINETOBACTER BAUMANNII-CALCOACETICUS COMPLEX

(71) Applicant: Entasis Therapeutics, Inc., Waltham, MA (US)

(72) Inventor: David Altarac, Waltham, MA (US)

(21) Appl. No.: 19/210,342

(22) Filed: May 16, 2025

(30) Foreign Application Priority Data
May 16, 2024 (WO) PCT/US2024/029741

(51) Int. Cl. A61K 31/551; A61K 31/43; A61P 31/04

(52) U.S. Cl. CPC A61K 31/551; A61K 31/43; A61P 31/04

(57) ABSTRACT

Provided are methods of treating hospital-acquired bacterial pneumonia and ventilator-associated bacterial pneumonia (HABP/VABP) caused by susceptible isolates of Acinetobacter baumannii-calcoaceticus complex using durlobactam and sulbactam.

### RELATED APPLICATIONS

This application claims the benefit of priority to international application No. PCT PCT/US2024/029741, filed May 16, 2024, the entire contents of which are incorporated herein by reference.

### BACKGROUND

Acinetobacter baumannii has emerged globally as a cause of many serious infections such as urinary tract infections, wound and surgical site infection, bacteremia, meningitis, and nosocomial infections, including ventilator-associated pneumonia (VAP). VAP is the most frequent A. baumannii infection in intensive care unit (ICU) patients, with a mortality rate of 25-75%. Chaari, et al., Acinetobacter baumannii Ventilator-Associated Pneumonia: Epidemiology, Clinical Characteristics, and Prognosis Factors, Int. J. Infectious Diseases, 17(12):e1225-e1228 (2013). About 63% of A. baumannii isolates are considered multi-drug resistant (MDR), which severely limits the treatment options, and which drives the high mortality rate. Karageorgopoulos, et al., Current Control and Treatment of Multi-Drug Resistant Acinetobacter Infections, Lancet, 8(12):751-762 (2008).

To help improve the effectiveness of β-lactam antibiotics, some β-lactamase inhibitors have been developed. However, typical β-lactamase inhibitors in many instances are insufficient to counter the constantly increasing diversity of β-lactamases. Most currently available β-lactamase inhibitors have activity primarily against certain Class A enzymes, which severely limits their utility. Additionally, new β-lactamase inhibitors, such as avibactam (approved in the US in 2015) and relebactam (MK-7655, still in clinical trials) work primarily on Class A and C enzymes, with minimal effectiveness against Class D β-lactamases. Bebrone, et al., Current Challenges in Antimicrobial Chemotherapy: Focus on β-Lactamase Inhibition, Drugs, 70(6):651-679 (2010).

Sulbactam is the Class A β-lactamase inhibitor (2S,5R)-3,3-dimethyl-7-oxo-4-thia-1-azabicyclo[3.2.0]heptane-2-carboxylic acid 4,4-dioxide. In addition to being a β-lactamase inhibitor, it also has intrinsic activity against a few pathogens, including Acinetobacter baumannii. Currently, sulbactam is commercially available in the United States in combination with ampicillin, which is marketed as Unasyn® and is approved in the US for treatment of skin, gynecological and intra-abdominal infections; it is also sold in the US as an oral agent Sultamicillin®. Adnan, et al., Ampicillin/Sulbactam: Its Potential Use in Treating Infections in Critically Ill Patients, Int. J. Antimicrobial Agents, 42(5):384-389 (2013). Clinically, Unasyn® has been used to treat VAP, bacteremia and other nosocomial infections caused by A. baumannii, even though ampicillin has no activity against the pathogen. However, significant resistance is emerging in the clinic. See, Jones, et al., Resistance Surveillance Program Report for Selected European Nations, Diagnostic Microbiology & Infectious Disease, 78(4): 429-436 (2011). Sulbactam is also commercially available in certain regions of the world in combination with cefoperasone and is sold as Cefina-SB®, Sulperazone® or Bacperazone®, depending on the geographic region.

While sulbactam is itself a β-lactamase inhibitor, it does not possess activity against many clinically relevant β-lactamases such as TEM-1 and Klebsiella pneumonia carbapenemases (KPCs), in addition to having no activity against most Class C and Class D β-lactamases. This upsurge in resistance means that sulbactam will have less and less clinical efficacy for patients with Acinetobacter spp. infections.

Given that the incidence of infections caused by MDR A. baumannii is increasing worldwide, the need exists for alternative therapies that effectively treat infections caused by such, particularly hospital-acquired bacterial pneumonia and ventilator-associated bacterial pneumonia (HABP/VABP).

### SUMMARY

Provided herein are methods of treating hospital-acquired bacterial pneumonia and ventilator-associated bacterial pneumonia (HABP/VABP) caused by susceptible isolates of Acinetobacter baumannii-calcoaceticus complex using durlobactam or a pharmaceutically acceptable salt thereof optionally in combination with sulbactam or a pharmaceutically acceptable salt thereof.

In some aspects, the period of administration and/or dosage of the effective amount of durlobactam or a pharmaceutically acceptable salt thereof, is adjusted based on the creatinine clearance rate of the subject.

In some aspects, the period of administration and/or dosage of the effective amount of sulbactam or a pharmaceutically acceptable salt thereof, is adjusted based on the creatinine clearance rate of the subject.

In some aspects, the HABP/VABP described herein is only caused by susceptible isolates of Acinetobacter baumannii-calcoaceticus complex.

Further provided are kits comprising durlobactam or a pharmaceutically acceptable salt thereof and sulbactam or a pharmaceutically acceptable salt thereof with Sodium Chloride Injection and Water for Injection.

### BRIEF DESCRIPTION OF THE FIGURES

FIG. 1 shows the trial design for 1.0 g sulbactam/1.0 g durlobactam for 7-14 days of treatment in patients with serious infections caused by ABC.

FIG. 2 shows the results from Phase 3 trial 28-day all-cause mortality for carbapenem-resistant Acinetobacter baumannii-calcoaceticus complex (CRABC microbiologically modified intent to treat (m-MITT) population.

FIG. 3 shows the results from Phase 3 trial 28-day and 14-day all-cause mortality for CRABC m-MITT, m-MITT, and ITT populations.

FIG. 4 shows the results from Phase 3 trial clinical cure rate at test of cure visit for CRABC m-MITT population.

FIG. 5 shows the microbiological favorable response at test of cure visit for CRABC m-MITT Population.

FIG. 6 shows the clinical cure rates and microbiological favorable responses at all measured timepoints for CRABC m-MITT Population.

FIG. 7 shows the 28-Day mortality, clinical cure rate at test of cure, and microbiological favorable assessment at test of cure for CRABC m-MITT Population.

FIG. 8 shows the incidence of nephrotoxicity as measured by modified RIFLE criteria for safety population excluding patients with chronic hemodialysis at baseline.

### DETAILED DESCRIPTION

Provided is a method of method of treating hospital-acquired bacterial pneumonia and ventilator-associated bacterial pneumonia (HABP/VABP) in a subject in need thereof, comprising administering to the subject an effective amount of durlobactam or a pharmaceutically acceptable salt thereof, wherein the HABP/VABP is caused by susceptible isolates of Acinetobacter baumannii-calcoaceticus complex.

Also provided is a method of method of treating hospital-acquired bacterial pneumonia and ventilator-associated bacterial pneumonia (HABP/VABP) in a subject in need thereof, comprising administering to the subject an effective amount of durlobactam or a pharmaceutically acceptable salt thereof, and an effective amount of sulbactam or a pharmaceutically acceptable salt thereof, wherein the HABP/VABP is caused by susceptible isolates of Acinetobacter baumannii-calcoaceticus complex.

Durlobactam can be prepared following the procedures described in e.g., WO 2013/150296, and refers to the chemical entity having the following structural formula:

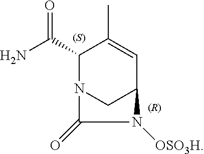

Durlobactam sodium, or the sodium salt of durlobactam refers to the chemical compound having the following structural formula:

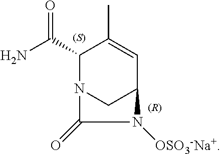

Sulbactam refers to (2S,5R)-3,3-dimethyl-7-oxo-4-thia-1-azabicyclo[3.2.0]heptane-2-carboxylic acid 4,4-dioxide, which is the chemical entity represented by the structure:

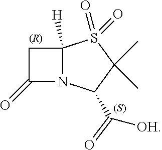

Sulbactam sodium, or the sodium salt of sulbactam refers to the chemical entity represented by the structure:

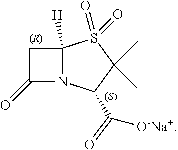

The terms “subject” and “patient” are used interchangeably and refer to a human. In some aspects, the subject is 18 years of age or older.

Treating, as used herein, refers to reversing, alleviating, or inhibiting the progress of HABP/VABP, or one or more symptoms thereof.

The term “effective amount” with respect to durlobactam, sulbactam, or the pharmaceutically acceptable salts thereof, means an amount of the compound or pharmaceutically acceptable salt thereof, which is sufficient enough to significantly and positively modify the symptoms of HABP/VABP and/or HABP/VABP (e.g., provide a positive clinical response). The effective amount of may depending upon the severity of the condition, the duration of the treatment, the nature of concurrent therapy, and like factors within the knowledge and expertise of the attending physician. The exact dose will depend on the purpose of the treatment, and will be ascertainable by one skilled in the art using known techniques (see, e.g., Lloyd (1999) The Art, Science and Technology of Pharmaceutical Compounding).

As used herein, “pharmaceutically acceptable salt” refers to those salts which are, within the scope of sound medical judgment, suitable for use in contact with the tissues of human beings and animals without excessive toxicity, irritation, allergic response, or other problem or complication, commensurate with a reasonable benefit/risk ratio. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2 hydroxyethyl¬sulfonate, heptanoate, hexanoate, hydrochloride, hydrobromide, hydroiodide, hydroxymaleate, lactate, malate, maleate, methanesulfonate, meglumine, 2 naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p toluenesulfonate), trifluoroacetate, and undecanoate. Examples of base salts include ammonium salts; alkali metal salts such as sodium, lithium and potassium salts; alkaline earth metal salts such as aluminum, calcium and magnesium salts; salts with organic bases such as dicyclohexylamine salts and N methyl D glucamine; and salts with amino acids such as arginine, lysine, ornithine, and so forth.

In certain aspects, durlobactam or a pharmaceutically acceptable salt thereof and/or sulbactam or a pharmaceutically acceptable salt thereof is administered to the subject intravenously.

In certain aspects, durlobactam or a pharmaceutically acceptable salt thereof and/or sulbactam or a pharmaceutically acceptable salt thereof is administered to the subject about every 4 hours. In certain aspects, durlobactam or a pharmaceutically acceptable salt thereof and/or sulbactam or a pharmaceutically acceptable salt thereof is administered to the subject about every 4 hours, wherein the subject has a creatinine clearance (CrCl) of greater than or equal to about 130 mL/min.

In certain aspects, durlobactam or a pharmaceutically acceptable salt thereof and/or sulbactam or a pharmaceutically acceptable salt thereof is administered to the subject about every 6 hours. In certain aspects, durlobactam or a pharmaceutically acceptable salt thereof and/or sulbactam or a pharmaceutically acceptable salt thereof is administered to the subject about every 6 hours, wherein the subject has a creatinine clearance (CrCl) of about 45 to about 129 mL/min.

In certain aspects, durlobactam or a pharmaceutically acceptable salt thereof and/or sulbactam or a pharmaceutically acceptable salt thereof is administered to the subject about every 8 hours. In certain aspects, durlobactam or a pharmaceutically acceptable salt thereof and/or sulbactam or a pharmaceutically acceptable salt thereof is administered to the subject about every 8 hours, wherein the subject has a creatinine clearance (CrCl) of about 30 to about 44 mL/min.

In certain aspects, durlobactam or a pharmaceutically acceptable salt thereof and/or sulbactam or a pharmaceutically acceptable salt is administered to the subject about every 12 hours. In certain aspects, durlobactam or a pharmaceutically acceptable salt thereof and/or sulbactam or a pharmaceutically acceptable salt thereof is administered to the subject about every 12 hours, wherein the subject has a creatinine clearance (CrCl) of about 15 to about 29 mL/min.

In certain aspects, durlobactam or a pharmaceutically acceptable salt thereof and/or sulbactam or a pharmaceutically acceptable salt thereof is administered intravenously to the subject over a period of about 3 hours.

In certain aspects, durlobactam or a pharmaceutically acceptable salt thereof and sulbactam or a pharmaceutically acceptable salt thereof are administered concurrently.

In certain aspects, the subject is administered durlobactam sodium. In certain aspects, the subject is administered sulbactam sodium.

In certain aspects, the subject is administered durlobactam sodium in an amount equivalent to about 1 gram of sulbactam. In certain aspects, the subject is administered sulbactam sodium in an amount equivalent to about 1 gram of sulbactam.

In certain aspects, the subject is treated for about 7 to about 14 days.

In certain aspects, the HABP/VABP treated by the described methods is caused only by susceptible isolates of Acinetobacter baumannii-calcoaceticus complex.

In certain aspects, the subject treated by the described methods is 18 years of age or older.

In certain aspects, concomitant administration of durlobactam or a pharmaceutically acceptable salt thereof and/or sulbactam or a pharmaceutically acceptable salt thereof with OAT1 inhibitors may increase plasma concentrations of sulbactam. As such concomitant administration of OAT1 inhibitors (e.g., probenecid) with durlobactam or a pharmaceutically acceptable salt thereof and/or sulbactam or a pharmaceutically acceptable salt thereof is not recommended.

Also provided is a kit comprising about 1 gram of sulbactam, about 1 gram of durlobactam, about a 100 mL infusion bag containing about 0.9% Sodium Chloride Injection, about 10 mL Sterile Water for Injection, a sterile syringe, and alcohol wipes.

### Exemplification

Treatment of Hospital-Acquired and Ventilator-Associated Bacterial Pneumonia Caused by Acinetobacter baumannii-Calcoaceticus Complex Organisms

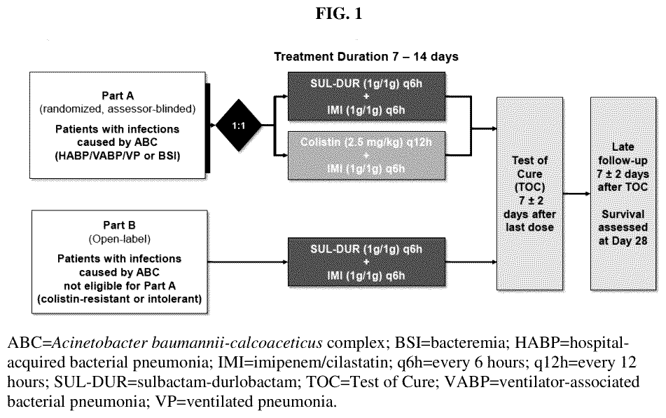

A global, two-part (A and B) Phase 3 trial was conducted that assessed the efficacy and safety of sulbactam/durlobactam in patients with serious infections caused by ABC, including multidrug- and carbapenem-resistant strains (FIG. 1). The Phase 3 trial employed a non-inferiority design, as discussed and agreed with the FDA DAI. A Test of Cure (TOC) visit was completed 7±2 days after the last dose, and survival was assessed at Day 28.

Patients were treated with either XACDURO® (1 g sulbactam and 1 g durlobactam, or renally adjusted dose) intravenously over 3 hours every 6 hours (n=91) or colistin 2.5 mg/kg (or renally adjusted dose) intravenously over 30 minutes every 12 hours after an initial loading dose of colistin 2.5 to 5 mg/kg (n=86). Both treatment arms also received 1 g imipenem/1 g cilastatin (or renally adjusted dose) intravenously every 6 hours as background therapy for potential HABP/VABP pathogens other than Acinetobacter baumannii-calcoaceticus complex. Patients received up to 14 days of therapy.

### Phase 3, Part A

Part A was a randomized, assessor-blinded, comparative portion of the Phase 3 trial. Patients were randomized (1:1) to 1.0 g sulbactam/1.0 g durlobactam or 2.5 mg/kg colistin. All patients in both treatment groups received 1.0 g imipenem/1.0 g cilastatin as background therapy to treat non-ABC co-infecting pathogens. Imipenem/cilastatin was also considered an effective therapy for patients with infections caused by carbapenem-susceptible ABC, an appropriate therapeutic partner to treat CRABC in the colistin group, and had a dosing regimen (q6h) consistent with sulbactam-durlobactam administration in patients with normal renal function.

Randomization was stratified by infection type (HABP/VABP/ventilated pneumonia [VP] vs bacteremia), severity of illness, and geography. A total of 92 patients were randomized to the sulbactam-durlobactam group and 89 patients to the colistin group.

Eligible patients were ≥18 years of age with a diagnosis of a serious infection caused by ABC as either a single pathogen or member of a polymicrobial infection confirmed by culture. In addition, patients must have had ≤48 hours of potentially effective antimicrobial therapy before first dose of study drug or clinically failed prior treatment (i.e., clinical deterioration or failure to improve after ≥48 hours of antibiotics) and an Acute Physiology and Chronic Health Evaluation (APACHE) II score 10-30 or Sequential Organ Failure Assessment (SOFA) score 1-11. Patients were excluded from Part A if they had an infection known to be resistant to colistin or polymyxin B. Other key exclusion criteria included hypersensitivity or allergic reaction to a β-lactam, contraindication to use of cilastatin, pulmonary disease that precludes evaluation of therapeutic response, and presence of suspected or confirmed deep-seated infection.

### Non-Inferiority Margin

A 20% non-inferiority margin for the primary efficacy endpoint for Part A was proposed. Based on literature review, the best estimate of the mortality rate for colistin-based therapy was 40% (95% CI: 35%, 45%) from a fixed effects analysis, or 40% (95% CI: 32%, 47%) from a random effects analysis using the method of DerSimonian and Laird (DerSimonian and Laird 1986). However, after updating the meta-analysis with 4 additional studies, the estimated mortality rate from the random effects meta-analysis was 41% (95% CI: 36%, 47%). The best estimate of the mortality rate for untreated or delayed treatment was 78% (95% CI: 72%, 83%) from a fixed effect analysis, or 76% (95% CI: 66%, 86%) from a random effects analysis.

Using the most conservative estimates of mortality from the updated meta-analysis, the mortality rate was estimated to be 41% (95% CI: 36%, 47%) for colistin-based therapy, and 76% (95% CI: 66%, 86%) for untreated or delayed therapy. Based upon these data and using the most conservative approach of taking the upper bound of the 95% CI from the colistin-based therapy estimate and the lower bound of the 95% CI from the inappropriate or delayed therapy estimate lead to an estimated treatment benefit of at least 19% (66% minus 47%; M1).

Given the unmet need of this population, the life-threatening condition, and the relevancy of the literature review study populations to this study population, clinically it was determined that it may not be necessary to preserve the entire 50% of the M1. FDA DAI independently determined that a 19% non-inferiority margin should be used; however, later evaluated again and agreed to a 20% margin for the study.

Estimation of the sample size assumed a 41% mortality rate in the colistin arm, a 36% mortality rate in the sulbactam-durlobactam arm, a 1:1 randomization, and an 80% power with a two-sided significance level of 0.05. The non-inferiority was based on the 2-sided 95% CIs computed using a continuity-corrected Z-statistic for the difference ([sulbactam-durlobactam]−[colistin]) in 28-day all-cause mortality rates between the treatment groups. Non-inferiority was concluded if the upper limit of the 2-sided 95% CI was <20%.

Secondary efficacy endpoints for Parts A and B included: 28-day all-cause mortality in the m-MITT and ITT Populations, 14-day all-cause mortality in the CRABC m-MITT and m-MITT Populations, Clinical cure at TOC, End of Treatment (EOT), and Late Follow-Up (LFU) in the CRABC m-MITT Population, and Microbiological favorable assessment at TOC, EOT, and LFU in the CRABC m-MITT Population.

In the ITT Population in Part A, 75% of patients in the sulbactam-durlobactam group and 68.5% of patients in the colistin group completed the trial. In the sulbactam-durlobactam group, the primary reasons patients discontinued were due to death (16.3%), no growth of ABC (2.2%), and patients who withdrew voluntarily (2.2%). In the colistin group, the primary reasons patients discontinued were due to death (23.6%) and patients who withdrew voluntarily (3.4%).

In the ITT Population in Part B, 78.6% of patients completed the trial. Four patients discontinued due to death, 1 patient discontinued due to withdrawal of consent, and 1 patient discontinued due to incorrect enrollment into Part B.

Primary Efficacy Endpoint Results—28-Day AU-Cause Mortality in Part A (CRABC m-MITT Population)

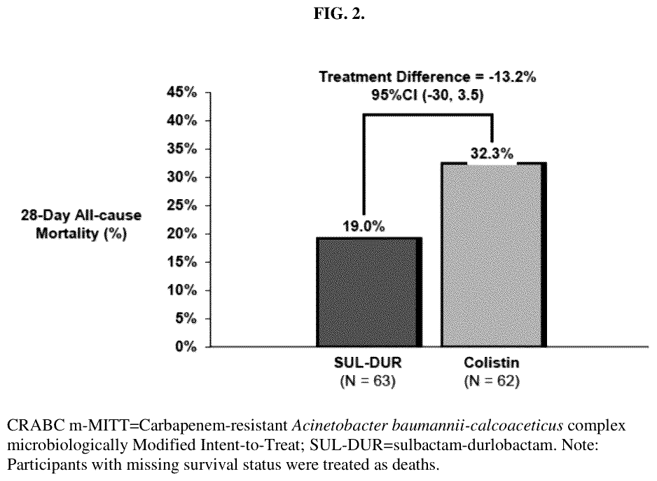

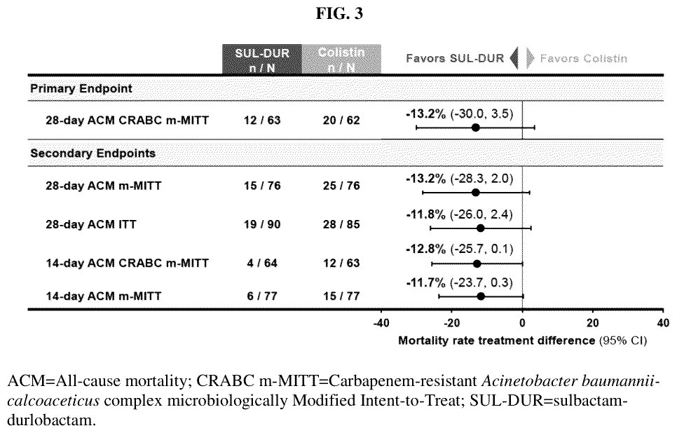

The Phase 3 trial met its prespecified primary efficacy endpoint for non-inferiority in Part A. In Part A of the CRABC m-MITT Population, the 28-day all-cause mortality for the sulbactam-durlobactam group was 19.0% (12/63 patients) and was 32.3% (20/62 patients) in the colistin group with a treatment difference of −13.2% (95% CI: −30.0%, 3.5%), which was less than the non-inferiority margin of 20% (FIG. 2 and FIG. 3). A test for superiority was conducted and the upper limit of the 2-sided CI was not <0.

### Secondary Efficacy Endpoint Results for Part A

Sulbactam-durlobactam met the secondary efficacy endpoints of all-cause mortality compared to colistin across various prespecified analysis populations at 28 days and 14 days (FIG. 3). All endpoints favored sulbactam-durlobactam.

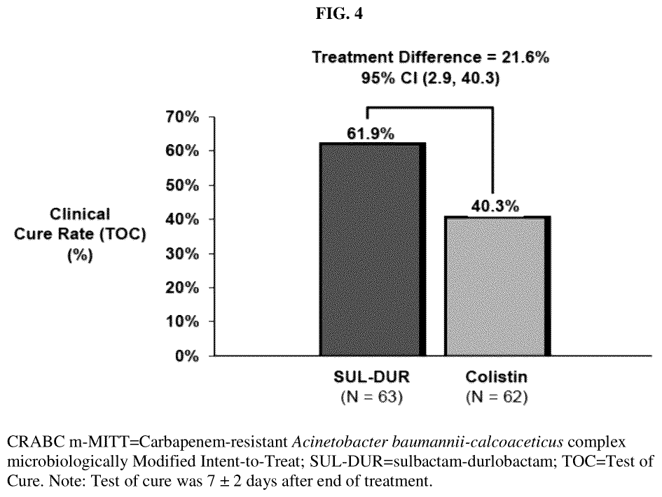

For Part A, a significant treatment difference of 21.6% (95% CI: 2.9%, 40.3%) in clinical cure rate at TOC was observed with 61.9% of patients in the sulbactam-durlobactam group compared to 40.3% of patients in the colistin group for the CRABC m-MITT Population (FIG. 4). Clinical cure was defined as complete resolution or significant improvement of baseline signs and symptoms and no new symptoms, such that no additional Gram-negative antimicrobial therapy was warranted.

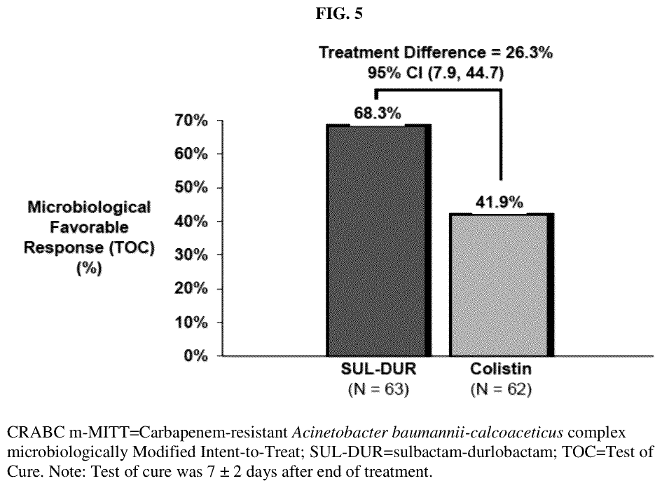

In addition, a significant treatment difference in microbiological favorable assessment at TOC was observed in the sulbactam-durlobactam group (68.3%) compared to the colistin group (41.9%) in the CRABC m-MITT Population, with a treatment difference of 26.3% (95% CI: 7.9%, 44.7%) (FIG. 5). A microbiological favorable assessment included eradication and presumed eradication.

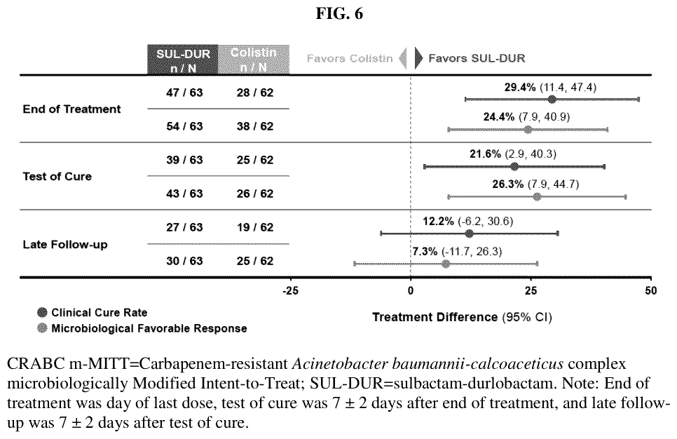

Clinical cure rates and microbiological responses were consistent between EOT and TOC for the CRABC m-MITT Population (FIG. 6). The difference between sulbactam/durlobactam versus colistin was lower in the late follow-up timepoint; however, sulbactam-durlobactam remained favorable for both clinical cure rate and favorable microbiological response.

### Secondary Efficacy Endpoint Results for Part B

Overall, results in Part B for patients who were intolerant to colistin or had infections caused by colistin-resistant ABC (N=28) were similar to patients in Part A treated with sulbactam-durlobactam.

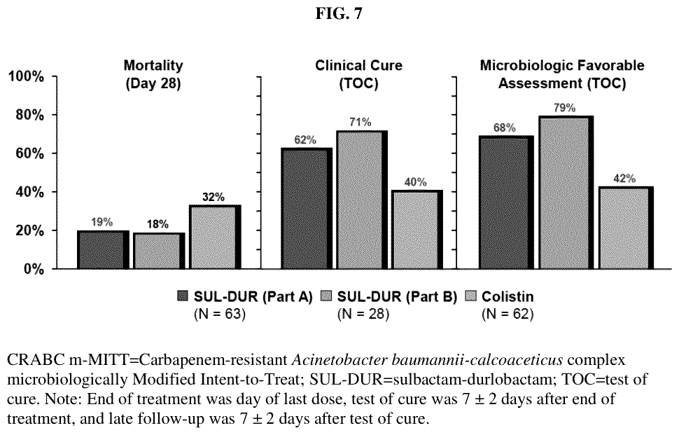

For Part B, 28-day all-cause mortality was 17.9% (5/28; 95% CI: 6.1%, 36.9%) for the ITT Population and was similar to the sulbactam-durlobactam group in Part A for the CRABC m-MITT Population (17.9% and 19.0%, respectively) (FIG. 7). In the 17 patients with bacteremia, 2 (11.8%) deaths occurred. Clinical cure at TOC was observed with 71.4% (20/28) of patients for the CRABC m-MITT Population (FIG. 7). Microbiological favorable assessment was observed at TOC in 78.6% of patients for the CRABC m-MITT Population (FIG. 7).

### Safety Findings

The cumulative safety data from the Phase 3 trial demonstrate that sulbactam/durlobactam is generally well tolerated in this critically ill patient population. The types and frequency of adverse events (AEs) reported were consistent with expectations for the patient population and were characteristic of the pharmacological class. Moreover, the overall incidence of treatment-emergent AEs (TEAEs), treatment-related AEs, severe TEAEs, serious TEAEs (SAEs), treatment-related SAEs, and TEAEs leading to study drug discontinuation or death were all lower in patients treated with sulbactam/durlobactam compared to those treated with colistin. In addition, the Phase 3 trial met the primary safety objective, with a statistically significant lower incidence of nephrotoxicity compared to colistin based on modified RIFLE criteria.

### Treatment Exposure

Overall, 380 individuals were been exposed to durlobactam alone or in combination with sulbactam in the sulbactam-durlobactam clinical development program. The proposed dose of 1.0 g sulbactam/1.0 g durlobactam q6h (adjusted for renal function) was administered to 181 individuals with 158 of those receiving sulbactam-durlobactam for the proposed duration of at least 7 days. Based on the integrated data of patients who received sulbactam-durlobactam at the proposed dose and duration, the mean duration of exposure to sulbactam-durlobactam was 9.1 days.

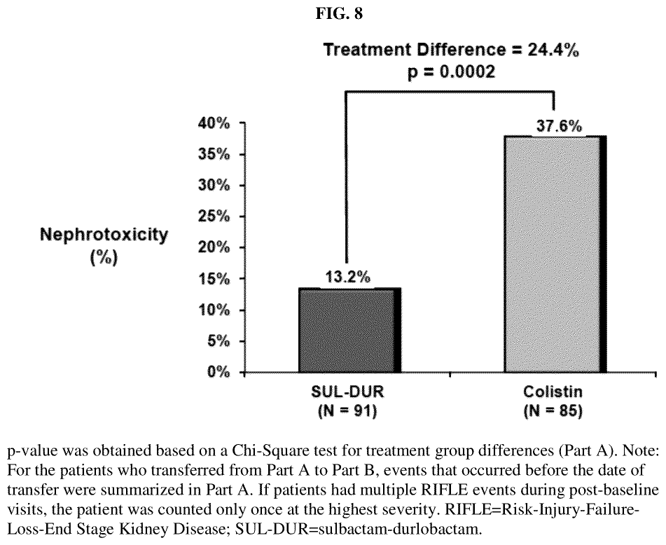

There was a significantly lower incidence of nephrotoxicity in the sulbactam-durlobactam group compared with the colistin group in Part A (13.2% vs 37.6%; p=0.0002; FIG. 8), based on modified Risk-Injury-Failure-Loss-End-Stage Kidney Disease (RIFLE) criteria as described by Hartzell J D, Neff R, Ake J, Howard R, Olson S, Paolino K, et al. Nephrotoxicity associated with intravenous colistin (colistimethate sodium) treatment at a tertiary care medical center. Clin Infect Dis. 2009; 48(12):1724-8. The modified RIFLE criteria includes: Risk (R): increased creatinine level 1.5× or glomerular filtration rate (GFR) decreased >25%, • Injury (I): increased creatinine level 2× or GFR decreased >50%, Failure (F): increased creatinine level 3×, GFR decreased >75%, or creatinine level ≥4 mg/dL, Loss (L): persistent acute renal failure or complete loss of function for >4 weeks, and End-Stage Kidney Disease (ESKD; E): ESKD for >3 months.

Consistent with the RIFLE assessment, the incidence and severity of AEs of renal and urinary disorders were lower in the sulbactam-durlobactam groups for Parts A and B compared to the colistin group. Nevertheless, the following doses are recommended based on renal function.

| Renal | Creatine | Sulbactam- | |
|---|---|---|---|
| Function | Clearance | Durlobactam | |
| Category | (mL/min)^{a} | Dose | Frequency of Dosing |
| Augmented | ≥130 to ≤200 | 1.0 g/1.0 g | q4h |
| renal | | | |
| clearance | | | |
| Normal and | ≥60 to <130 | 1.0 g/1.0 g | q6h |
| mild | | | |
| Moderate | ≥45 to <60 | 1.0 g/1.0 g | q6h |
| | ≥30 to <45 | 1.0 g/1.0 g | q8h |
| Severe | ≥15 to <30 | 1.0 g/1.0 g | q12h |
| | ≥0 to <15^{b} | 1.0 g/1.0 g | For initiation of treatment: |
| | | | Every 12 hours for the first |
| | | | 3 doses (0, 12, and 24 |
| | | | hours), followed by every |
| | | | 24 hours after the third |
| | | | dose^{c} |
| | | | For all other treatment |
| | | | course: |
| | | | Every 24 hours |

^{a}Creatinine clearance estimated by Cockcroft-Gault equation.
^{b}For patients on hemodialysis, the dose should be administered after the dialysis session has ended.
^{c}This is equivalent to 1.0 g sulbactam/1.0 g durlobactam q12h on Day 1 followed by 1.0 g sulbactam/1.0 g durlobactam every 24 hours.
qXh = every X hours

1. A method of treating hospital-acquired bacterial pneumonia and ventilator-associated bacterial pneumonia (HABP/VABP) in a subject in need thereof, comprising administering to the subject an effective amount of durlobactam or a pharmaceutically acceptable salt thereof, wherein the HABP/VABP is caused by susceptible isolates of Acinetobacter baumannii-calcoaceticus complex.

2. The method of claim 1, further comprising administering an effective amount of sulbactam or a pharmaceutically acceptable salt thereof.

3. The method of claim 1, wherein the durlobactam or a pharmaceutically acceptable salt thereof is administered intravenously.

4. The method of claim 1, wherein the durlobactam or a pharmaceutically acceptable salt thereof is administered to the subject about every 4 hours.

5. The method of claim 1, wherein the subject has a creatinine clearance (CrCl) of greater than or equal to about 130 mL/min.

6. The method of claim 1, wherein the durlobactam or a pharmaceutically acceptable salt thereof is administered to the subject about every 6 hours.

7. The method of claim 6, wherein the subject has a creatinine clearance (CrCl) of about 45 to about 129 mL/min.

8. The method of claim 1, wherein the durlobactam or a pharmaceutically acceptable salt thereof is administered to the subject about every 8 hours.

9. The method of claim 8, wherein the subject has a creatinine clearance (CrCl) of about 30 to about 44 mL/min.

10. The method of claim 1, wherein the durlobactam or a pharmaceutically acceptable salt thereof is administered to the subject about every 12 hours.

11. The method of claim 10, wherein the subject has a creatinine clearance (CrCl) of about 15 to about 29 mL/min.

12. The method of claim 1, wherein the durlobactam or a pharmaceutically acceptable salt thereof is administered intravenously to the subject over a period of about 3 hours.

13. The method of claim 1, wherein the sulbactam or a pharmaceutically acceptable salt thereof is administered intravenously.

14. The method of claim 1, wherein the sulbactam or a pharmaceutically acceptable salt thereof is administered to the subject about every 4 hours.

15. The method of claim 14, wherein the subject has a creatinine clearance (CrCl) of greater than or equal to about 130 mL/min.

16. The method of claim 1, wherein the sulbactam or a pharmaceutically acceptable salt thereof is administered to the subject about every 6 hours.

17. The method of claim 16, wherein the subject has a creatinine clearance (CrCl) of about 45 to about 129 mL/min.

18. The method of claim 1, wherein the sulbactam or a pharmaceutically acceptable salt thereof is administered to the subject about every 8 hours.

19. The method of claim 18, wherein the subject has a creatinine clearance (CrCl) of about 30 to about 44 mL/min.

20. The method of claim 1, wherein the sulbactam or a pharmaceutically acceptable salt thereof is administered to the subject about every 12 hours.

21. The method of claim 20, wherein the subject has a creatinine clearance (CrCl) of about 15 to about 29 mL/min.

22. The method of claim 1, wherein the sulbactam or a pharmaceutically acceptable salt thereof is administered intravenously to the subject over a period of about 3 hours.

23. The method of claim 1, wherein the durlobactam or a pharmaceutically acceptable salt thereof and sulbactam or pharmaceutically acceptable salt thereof are administered concurrently.

24. The method of claim 1, wherein the subject is administered durlobactam sodium.

25. The method of claim 1, wherein the subject is administered durlobactam sodium in an amount equivalent to about 1 gram of durlobactam.

26. The method of claim 1, wherein the subject is administered sulbactam sodium.

27. The method of claim 1, wherein the subject is administered sulbactam sodium in an amount equivalent to about 1 gram of sulbactam.

28. The method of claim 1, wherein the subject is treated for about 7 to about 14 days.

29. The method of claim 1, wherein the HABP/VABP is caused only by susceptible isolates of Acinetobacter baumannii-calcoaceticus complex.

30. The method of claim 1, wherein the subject is 18 years of age or older.

31. A kit comprising about 1 gram of sulbactam, about 1 gram of durlobactam, about a 100 mL infusion bag containing about 0.9% Sodium Chloride Injection, about 10 mL Sterile Water for Injection, a sterile syringe, and alcohol wipes.
